(19) United States
(12) Patent Application Publication
(10) Pub. No.: US 2022/0167860 A1
(43) Pub. Date: Jun. 2, 2022

(54) BLOOD PRESSURE MEASUREMENT SYSTEM AND BLOOD PRESSURE MEASUREMENT METHOD USING SAME

(71) Applicant: CHARMCARE CO., LTD., Seoul (KR)

(72) Inventor: Dong Hwa LEE, Yongin-si (KR)

(21) Appl. No.: 17/436,325

(22) PCT Filed: Feb. 26, 2020
(86) PCT No.: PCT/KR2020/002728

§ 371 Date: Sep. 3, 2021

(30) Foreign Application Priority Data
Mar. 6, 2019 (KR) 10-2019-0025830

(51) Int. Cl. A61B 5/021; A61B 5/053

(52) U.S. Cl. CPC A61B 5/02108; A61B 5/02141; A61B 2562/0247; A61B 2562/0219; A61B 5/053

(57) ABSTRACT

Provided is a blood pressure measurement system including a pulse wave measurement sensor unit that measures an arterial wave from an artery, and a blood pressure calculation unit that calculates blood pressure from an arterial wave detected by the pulse wave measurement sensor unit, and a blood pressure measurement method using the blood pressure measurement system. The pulse wave measurement sensor unit measures a first arterial wave under a constant pressure and measures a second arterial wave under a variable pressure, and the blood pressure calculation unit calculates a mapped arterial wave by mapping the first arterial wave measured under the constant pressure to the second arterial wave measured under the variable pressure and calculates blood pressure by using the mapped arterial wave.

### TECHNICAL FIELD

The present invention relates to a blood pressure meter and a blood pressure measurement method, and more particularly, to a blood pressure measurement system capable of detecting an arterial wave of at least one cycle to calculate a blood pressure value only with an arterial wave of one cycle at high speed, and a blood pressure measurement method using the blood pressure measurement system.

### BACKGROUND

In general, pressure of blood applied to a wall of blood vessel is called blood pressure, and the heart repeats contraction and relaxation about 60 to about 80 times per minute. Pressure of blood vessels at the time when the heart contracts and pushes blood, is called a “systolic blood pressure” and is also called a “maximal blood pressure” because the blood pressure is the highest. In addition, a blood vessel pressure at the time when the heart relaxes and accepts blood is called a “diastolic blood pressure” and is also called a “minimal blood pressure” because the blood pressure is the lowest.

A systolic blood pressure of a normal person is 120 mmHg and a diastolic blood pressure thereof is 80 mmHg. More than one of four Korean adults has a high blood pressure, and after the age of 40, a ratio of the high blood pressure rapidly increases. In contrast to this, there are some patients classified as a low blood pressure.

When a high blood pressure is left uncontrolled, the high blood pressure can cause other life-threatening complications such as eye disease, kidney disease, arterial disease, brain disease, and heart disease. Therefore, in the case of patients at risk of complications or having complications, continuous blood pressure measurement and management have to be performed.

Various types of blood pressure measurement devices are developed as interest in health and diseases related to adult diseases such as hypertension is increased. A blood pressure measurement method includes an auscultation (Korotkoff sounds) method, an oscillometric method, a tonometric method, and so on.

The auscultation method is a general pressure measurement method, and is a method of measuring pressure at the moment when a pulse sound is first heard as a systolic blood pressure (systolic pressure) in the process of reducing the pressure after applying a sufficient pressure to a region of the body through which the arterial blood passes and blocking flow of blood, and measuring pressure at the moment when the pulse sound disappears as a diastolic blood pressure (diastolic pressure).

In addition, the oscillometric method and the tonometric method are applied to a digitized blood pressure measurement device. In the oscillometric method, a systolic blood pressure and a diastolic blood pressure are measured by detecting a pulse wave generated in the process of reducing pressure at a constant rate after sufficiently pressurizing a region of the body through which an arterial blood passes so as to block a blood flow of arteries, or a process of pressurizing the region of the body to increase pressure at a constant speed, like the auscultation method.

Here, pressure of a constant level can be measured as a systolic blood pressure or a diastolic blood pressure compared to the moment when an amplitude of a pulse wave is maximum, and pressure at the time when a change rate of the amplitude of the pulse wave is rapidly changed can also be measured as the systolic blood pressure or the diastolic blood pressure.

In the process of reducing pressure at a constant rate after pressurization, the systolic blood pressure is measured a certain time before the moment when the amplitude of the pulse wave is maximum, and the diastolic blood pressure is measured a certain time later than the moment when the amplitude of the pulse wave is maximum. In contrast to this, in the process of increasing pressure at a constant speed, the systolic blood pressure is measured later than the moment when the amplitude of the pulse wave is maximum, and the diastolic blood pressure is measured before the moment when the amplitude of the pulse wave is maximum.

The tonometric method is a method in which a certain pressure of a magnitude that does not completely block an arterial blood flow is applied to a region of the body, and blood pressure can be continuously measured by using a magnitude and a shape of the generated pulse wave.

As described above, the device for measuring blood pressure in various ways, that is, a blood pressure meter is the most basic medical device for measuring blood pressure which is the basis of a health index and are provided in general hospitals as an essential device and are often used to measure an individual blood pressure in homes or sports centers.

Most of the currently used blood pressure meters are designed to measure blood pressure on the upper arm which is similar to a height of the heart, and for the sake of convenient carry and measurement, products capable of measuring blood pressure in regions of the body such as fingers are also developed. The aforementioned wrist blood pressure meter or finger blood pressure meter has advantages of being convenient to carry and easy to measure at any time because of a small size compared to the upper arm blood pressure meter.

Meanwhile, a blood pressure meter of the related art that measures blood pressure by using arterial waves, for example, an oscillometric blood pressure meter measures blood pressure by detecting arterial pulses, that is, arterial waves of several cycles, and time for measuring blood pressure takes 40 seconds or more.

### SUMMARY OF INVENTION

### Technical Problem

The present invention relates to a blood pressure meter for measuring blood pressure and provides a blood pressure measurement system capable of detecting an arterial wave signal of at least one cycle from multiple types, for example, two types of arterial waves to calculate a blood pressure value only with an arterial wave of one cycle at high speed, and a blood pressure measurement method using the blood pressure measurement system.

### Solution to Problem

According to one embodiment of the present invention, a blood pressure measurement system includes a pulse wave measurement sensor unit which measures arterial waves, and a blood pressure calculation unit which calculates blood pressure from the arterial wave detected by the pulse wave measurement sensor unit, wherein the pulse wave measurement sensor unit measures one arterial wave under a constant pressure and measures another arterial wave under a variable pressure, and the blood pressure calculation unit calculates a mapped arterial wave by mapping a first arterial wave measured under the constant pressure to a second arterial wave measured under the variable pressure and calculates blood pressure by using the mapped arterial wave.

The pulse wave measurement sensor unit can include a first sensor which measures the first arterial wave, and a second sensor which measures the second arterial wave.

The blood pressure measurement system can further include a pressurization unit which applies pressure to a region where arterial wave measurement is performed by the second sensor. The pressurization unit can include any one of a tightening device which tightens a portion to be tested, an air pump which injects air into an air bag, a thermal expansion member, and a shape memory alloy.

The pressurization unit can further include a valve which opens and closes at least one of a passage for guiding air to the air bag and an air outlet for discharging air from the air bag.

The second sensor can measure the second arterial wave during one of a pressure increase process and a pressure reduction process. More specifically, the second sensor can measure the second arterial wave during a process in which pressure is increased or reduced at a constant rate.

Each of the first sensor and the second sensor can include any one of a pressure sensor, an optical sensor, and an impedance sensor which measures impedance of a blood vessel. Here, the pressure sensor can include any one of a pneumatic sensor, a film-type pressure sensor, and a strain meter.

The first sensor and the second sensor can respectively and simultaneously measure the first arterial wave and the second arterial wave at different positions.

The blood pressure calculation unit can calculate the mapped arterial wave by mapping the first arterial wave to the second arterial wave based on an arterial wave block time when the second arterial wave is measured. More specifically, the blood pressure calculation unit can determine a highest value of the mapped arterial wave as a maximal blood pressure and determines a lowest value of the mapped arterial wave as a minimal blood pressure.

According to another embodiment of the present invention, a blood pressure measurement method performed by a blood pressure measurement system including a pulse wave measurement sensor unit for detecting an arterial wave, includes a blood pressure calculation step of calculating a mapped arterial wave by mapping a first arterial wave measured under an isobaric pressure to a second arterial wave measured under a variable pressure by using a processor for calculating blood pressure, and calculating the blood pressure from the mapped arterial wave by using the processor.

The blood pressure measurement method can further include an arterial wave measurement step of simultaneously measuring the first arterial wave and the second arterial wave at different positions by using the pulse wave measurement sensor unit.

In the arterial wave measurement step, the second arterial wave can be measured during one of a pressure increase process and a pressure reduction process of a region where the second arterial wave is measured. More specifically, in the arterial wave measurement step, the second arterial wave can be measured during a process in which pressure of a region where the second arterial wave is measured is increased or reduced at a constant rate.

The mapped arterial wave can be calculated by mapping the first arterial wave to the second arterial wave based on an arterial wave block time when the second arterial wave is measured. More specifically, in the blood pressure calculation step, a highest value of the mapped arterial wave can be determined as a maximal blood pressure, and a lowest value of the mapped arterial wave can be determined as a minimal blood pressure.

### Advantageous Effects

According to the present invention, a blood pressure value can be calculated from two arterial waves detected in different regions and output, and thus, compared to the oscillometric blood pressure meter of the related art that takes 40 seconds or more to measure blood pressure, blood pressure can be quickly calculated from only one or more arterial waves, particularly, arterial waves of only one cycle, time taken to calculate the blood pressure can be greatly reduced, a complex blood pressure calculation algorithm can not be required, and a blood pressure calculation method can be simplified.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating a configuration of a blood pressure measurement system according to the present invention;

FIG. 2 is a diagram schematically illustrating an embodiment of the blood pressure measurement system according to the present invention;

FIG. 3 is a view illustrating a blood pressure measurement method performed by the blood pressure measurement system illustrated in FIG. 2;

FIG. 4 is a view schematically illustrating another embodiment of the blood pressure measurement system according to the present invention;

FIG. 5 is a view illustrating a blood pressure measurement method by the blood pressure measurement system illustrated in FIG. 4;

FIG. 6 is a view schematically illustrating another embodiment of the blood pressure measurement system according to the present invention;

FIG. 7 is a view illustrating a blood pressure measurement method performed by the blood pressure measurement system illustrated in FIG. 6;

FIG. 8 is a view schematically illustrating another embodiment of the blood pressure measurement system according to the present invention;

FIG. 9 is a view schematically illustrating another embodiment of the blood pressure measurement system according to the present invention;

FIG. 10 is a flowchart schematically illustrating a blood pressure measurement method according to an embodiment of the present invention; and

FIG. 11 is a graph illustrating the blood pressure measurement method according to the present invention.

### BEST MODE

Hereinafter, preferred embodiments of the present invention in which objects of the present invention can be specifically realized are described with reference to the accompanying drawings. In describing the present embodiments, the same names and reference numerals are used for the same components, and additional descriptions thereof are omitted below.

Terms used in the present specification are used to describe embodiments of the present invention and are not intended to limit the present invention. For example, terms including an ordinal number, such as “first” and “second”, can be used to distinguish components of the same name from each other, but do not define or limit the number of components.

In addition, when it is described that a component is “connected” or “coupled” to another component, it should be understood that the component can be directly connected or coupled to another component and the connection or coupling also includes a connection relationship in which other components exist therebetween, that is, a relationship that is indirectly connected.

In the present specification, it should be understood that terms such as “include” or “have” indicate that features, numbers, steps, operations, configuration elements, components, or combinations thereof described in the specification exist, and existence or addition of one or more other features, numbers, steps, operations, configuration elements, components, or combinations thereof are not excluded.

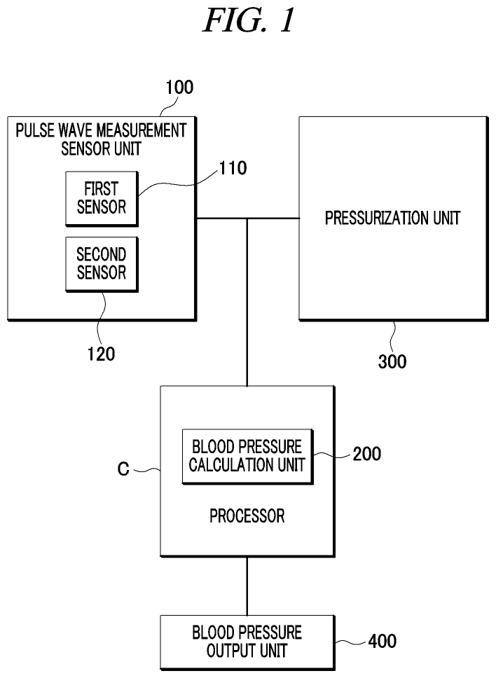

Referring to FIGS. 1 to 9, embodiments of the present invention relate to a blood pressure measurement system including a pulse wave measurement sensor unit 100 that measures an arterial wave from an artery, and a blood pressure calculation unit 200 that calculates blood pressure from an arterial wave detected by the pulse wave measurement sensor unit 100, and to a blood pressure measurement method using the blood pressure measurement system. The pulse wave measurement sensor unit 100 detects a plurality of arterial waves, for example, two arterial waves from an artery. The blood pressure calculation unit 200 calculates blood pressure by using different arterial waves, for example, two arterial waves to be described below which are detected by the pulse wave measurement sensor unit 100.

In embodiments of the present invention, the pulse wave measurement sensor unit 100 measures one arterial wave under a constant pressure (in a state where there is no external force applied to the artery or in a constant state), and measures another arterial wave under a variable pressure, that is, a pressure change environment (in a state in which an external force applied to the artery changes). For example, the pulse wave measurement sensor unit 100 simultaneously detects an arterial wave (first arterial wave) measured under an isobaric pressure and an arterial wave (second arterial wave) measured under variable pressure. That is, the pulse wave measurement sensor unit detects a plurality of arterial waves under different environments.

The pulse wave measurement sensor unit 100 detects an arterial wave in a certain region of the body. More specifically, the pulse wave measurement sensor unit 100 can include a first sensor 110 that measures the first arterial wave described above and the second sensor 120 that measures the second arterial wave.

The first sensor 110 and the second sensor 120 respectively and simultaneously measure the first arterial wave and the second arterial wave at different positions of the body. For example, the first sensor 110 detects an arterial wave, that is, the first arterial wave of a corresponding position in a state in which the first sensor 110 is in contact with a skin under a constant pressure. In addition, the second sensor 120 detects an arterial wave (the second arterial wave) at a position different from a measurement position of the first sensor 110. In this case, the second sensor 120 detects the second arterial wave in an environment in which the variable pressure, that is, a force (pressure) pressing the measurement position by the second sensor is changed.

The first sensor 110 and the second sensor 120 can include any one of an optical sensor such as a pressure sensor and an optical blood flow meter (a photoplethysmogram (PPG) sensor) and an impedance sensor for measuring impedance of a blood vessel. Here, the pressure sensor can include any one of a pneumatic pressure sensor and a film type pressure sensor. The above-described sensors are known, and thus, additional descriptions thereof are omitted.

The blood pressure calculation unit 200 maps the first arterial wave measured under an isobaric pressure to the second arterial wave measured under a variable pressure to calculate (obtain) a mapped arterial wave and calculates blood pressure by using the mapped arterial wave.

The blood pressure measurement system 10 can further include a pressurization unit 300 that applies pressure to a region (a measurement position of the second sensor) where arterial wave measurement is performed by the second sensor 120. As in the first embodiment to be described below, a variable pressure environment can be implemented manually as an examinee slowly pressurizes a region to be measured by the second sensor or reduces a pressing force, and a variable pressure can also be implemented automatically by the pressurization unit 300.

The pressurization unit 300 can include any one of a tightening device for tightening a portion to be inspected (for example, a wrist tightening device such as the examples disclosed in Patent Publication No. 10-2018-0019325 and Patent Publication No. 10-2017-0042118), an air pump for injecting air into an air bag 310, a thermal expansion member, and a shape memory alloy.

The pressurization unit 300 can further include a valve (not illustrated) for opening or closing at least one of a passage for guiding air to the air bag 310 and an air outlet for discharging air of the air bag.

The second sensor 120 can measure the second arterial wave during a pressure increase process or a pressure reduction process of the pressurization unit 300. The second sensor 120 can measure the second arterial wave during the pressure increase process or the pressure reduction process of the pressurization unit 300 at a constant rate. For example, while the air bag 310 is gradually inflated by an air pump or air is gradually discharged from the air bag 310 inflated by the air pump, measurement of the second arterial wave is made by the second sensor 120.

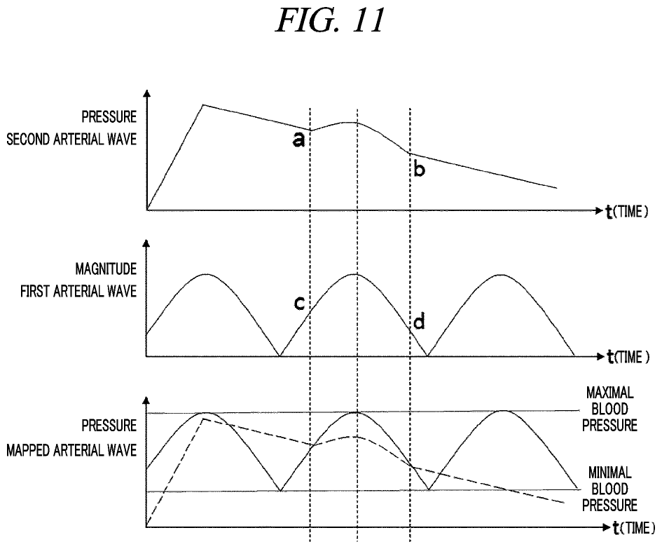

The blood pressure calculation unit 200 maps an arterial wave (the first arterial wave) measured under an isobaric pressure to another arterial wave (the second arterial wave) measured under a variable pressure based on an arterial wave block time (times of points a and b of an upper graph of graphs illustrated in FIG. 11) when measuring the second arterial wave to calculate the mapped arterial wave, and calculates blood pressure by using the mapped arterial wave. More specifically, the blood pressure calculation unit determines a highest value of the mapped arterial wave as a maximal blood pressure and determines a lowest value of the mapped arterial wave as a minimal blood pressure.

The pulse wave measurement sensor unit 100, that is, the first sensor 110 and the second sensor 120 can be controlled by a processor, that is, a controller C, and the pressurization unit 300 can also be controlled by controller C, and thereby, filling and exhausting of an air bag to be described below can also be performed. In addition, the blood pressure values calculated by the above-described method, for example, the maximal blood pressure and the minimal blood pressure are displayed on a blood pressure output unit 400 such as a digital monitor.

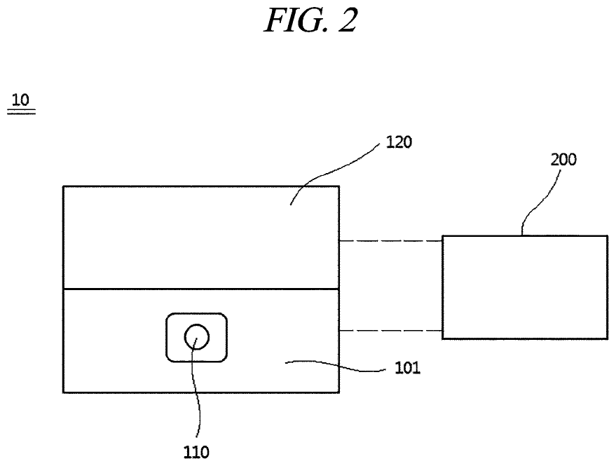

Hereinafter, specific embodiments of the blood pressure measurement system according to the present invention will be described with reference to FIGS. 2 to 9.

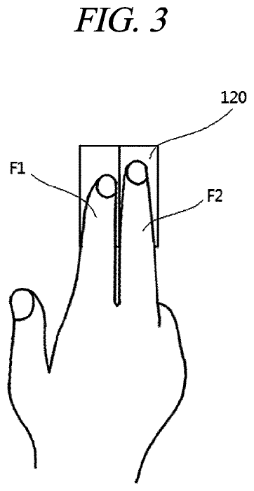

First, referring to FIGS. 2 and 3, a first embodiment 10 of the blood pressure measurement system according to the present invention includes an example in which the blood pressure measurement system is a blood pressure meter that detects a pulse wave of an artery, that is, an arterial wave from a finger, the first sensor 110 is configured with an optical sensor, and the second sensor 120 is configured with a film-type pressure sensor. The first sensor 110 can be placed on a finger pad 101.

An examinee puts one finger F1 on a position of the first sensor 110 (an optical sensor) to come into contact with the first sensor in a constant pressure and presses slowly and strongly a position of the second sensor 120 (a film-type pressure sensor) with another finger F2. During this process, the first sensor 110 detects a first arterial wave under an isobaric pressure, and the second sensor 120 detects a second arterial wave (a variable pressure arterial wave) under a variable pressure.

The finger pad 101 can also be provided in a band type that can be fixed by being wound around a finger, and the second sensor 120 can also be fixed to a finger in a band type.

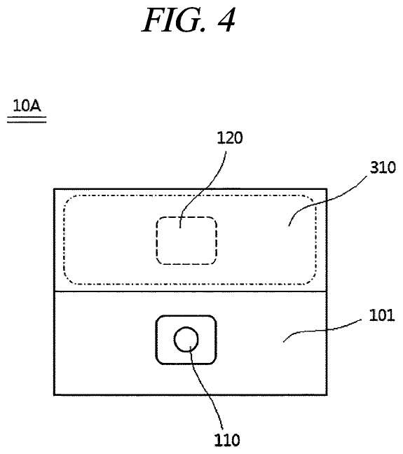

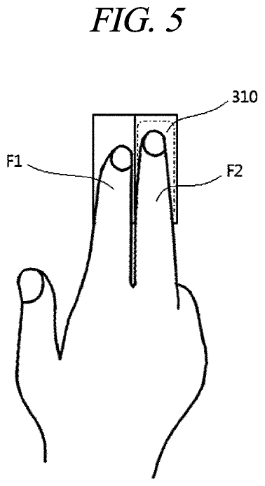

Next, referring to FIGS. 4 and 5, the second embodiment 10A of the blood pressure measurement system according to the present invention is an example in which the blood pressure measurement system is a blood pressure meter that detects an arterial wave from a finger, the first sensor 110 is configured with an optical sensor, and the second sensor 120 is configured with a pneumatic sensor, and the second sensor 120 is included in the air bag 310. The first sensor 110 and the second sensor 120 can be fixed by being wound around a finger in a band type as in the above-described embodiment.

An examinee puts one finger F1 on a portion of the first sensor 110 (an optical sensor) to cause the finger F1 to come into contact the first sensor in a constant pressure and presses the air bag 310 on which the second sensor 120 (a pneumatic sensor) is placed with another finger F2. The air bag 310 is filled with air, and the examinee presses the air bag 310 to a preset pressure, for example, 300 mmHg, with another finger F2 such that air is discharged through an air hole 311 of the air bag 310, and during the discharging process (a pressure reduction process), a variable pressure arterial wave, that is, the second arterial wave is detected by the second sensor 120 (pneumatic sensor).

When the first arterial wave and the second arterial wave (variable pressure arterial wave) are measured according to the first embodiment 10 and the second embodiment 10A described above, the blood pressure calculation unit 200 maps an arterial wave (the first arterial wave) measured under an isobaric pressure to an arterial wave (the second arterial wave) measured under a variable pressure based on an arterial wave block time when measuring the second arterial wave to calculate the mapped arterial wave, and calculates blood pressure by using the mapped arterial wave.

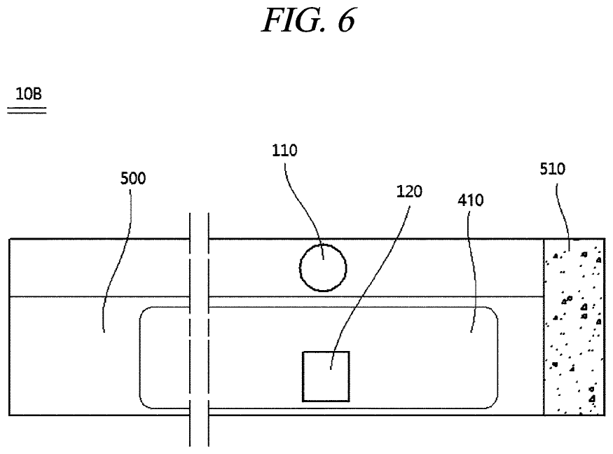

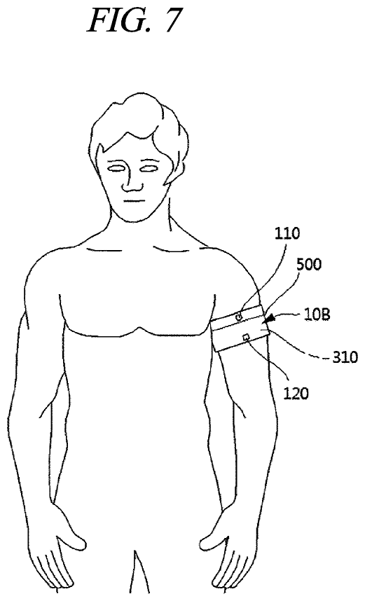

Referring to FIGS. 6 and 7, a third embodiment 10B of the blood pressure measurement system according to the present invention is an example in which the blood pressure measurement system is an upper arm cuff-type blood pressure meter and includes the first sensor 110 for detecting the first arterial wave and the second sensor 120 for detecting the second arterial wave, the first sensor 110 is configured with an optical sensor, and the second sensor 120 is configured with a pneumatic sensor.

The first sensor 110 and the second sensor 120 are provided on a cuff belt 500 worn on an upper arm. More specifically, the cuff belt 500 includes the air bag 310, and the air bag 310 can be filled with air by a manual or automatic pumping mechanism (an air pump). In addition, the second sensor 120, that is, the pneumatic sensor is included in the air bag 310, and the first sensor 110 is placed an external region of the air bag 310, that is, a region that is not affected by pressure of the air bag 310.

After the upper arm cuff-type blood pressure meter is worn on an examinee's upper arm by using belt fixing means such as a Velcro 510 called a hook and loop fastener or a button provided in the cuff belt 500, the air bag 310 is filled with air to a preset pressure to press the examinee's upper arm. Thereafter, the pressure is gradually reduced at a certain rate by exhaust of the air bag 310, and during the exhaust process, the first sensor 110 detects the first arterial wave (an optical arterial wave) under a constant pressure, and at the same time, the second sensor 120 (a pneumatic sensor) detects a variable pressure arterial wave, that is, the second arterial wave.

In addition, when the first arterial wave and the second arterial wave (a variable pressure arterial wave) are measured according to the third embodiment in the above-described manner, the blood pressure calculation unit 200 maps an arterial wave (the first arterial wave) measured under an isobaric pressure to an arterial wave (the second arterial wave) measured under a variable pressure based on the arterial wave block time when measuring the second arterial wave to calculate the mapped arterial wave, and calculates blood pressure by using the mapped arterial wave.

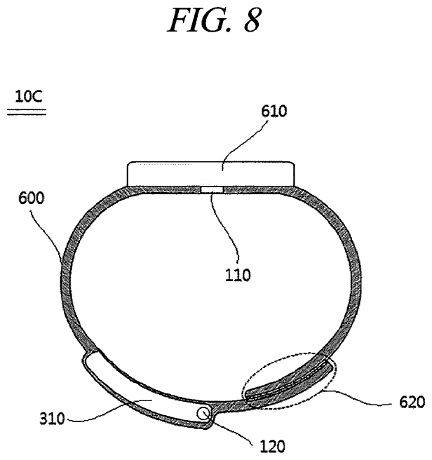

Referring to FIG. 8, a fourth embodiment of the blood pressure measurement system according to the present invention is an example in which the blood pressure measurement system is a wrist blood pressure meter 10C and includes the first sensor 110 for detecting a first arterial wave and the second sensor 120 for detecting a variable pressure arterial wave, that is, the second arterial wave, the first sensor 110 is configured with an optical sensor, and the second sensor 120 is configured with a pneumatic sensor.

The first sensor 110 and the second sensor 120 are provided in a wrist cuff 600 worn on the wrist. More specifically, the wrist cuff 600 includes the air bag 310, and the air bag 310 can be filled with air by a manual or automatic pumping mechanism (air pump). In addition, the second sensor 120, that is, the pneumatic sensor is provided in the air bag 310, and the first sensor 110 is provided in an external region area of the air bag 310, that is, a region that is not affected by pressure of the air bag 310, for example, a lower side of a case 610 for a display device (blood pressure output unit) that outputs a blood pressure value. The wrist cuff 600 is connected to be integrated by a strap attachment/detachment means 620 such as a Velcro, a button, or a buckle.

After the wrist blood pressure meter 10B is worn on an examinee's wrist, the air bag 310 is filled with air to a preset pressure to locally compress (for example, compress a region through which a radial artery or an ulnar artery passes) the examinee's wrist. Thereafter, pressure is gradually reduced at a certain rate by exhaust of the air bag 310, and during the exhaust process, the first sensor 110 detects the first arterial wave (an optical arterial wave) under a certain pressure, and at the same time, the second sensor 120 (a pneumatic sensor) detects a variable pressure arterial wave, that is, the second arterial wave.

In addition, when the first arterial wave and the second arterial wave (variable pressure arterial wave) are measured according to the fourth embodiment in the above-described manner, the blood pressure calculation unit 200 maps an arterial wave (the first arterial wave) measured under an isobaric pressure to an arterial wave (the second arterial wave) measured under a variable pressure based on an arterial wave block time when measuring the second arterial wave to calculate the mapped arterial wave, and calculates blood pressure by using the mapped arterial wave.

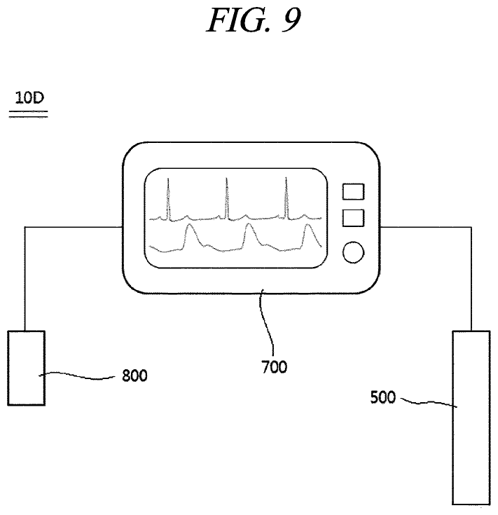

Next, referring to FIG. 9, a fifth embodiment 10D of the blood pressure measurement system according to the present invention is a blood pressure measurement system implemented as a patient monitoring device and includes an oxygen saturation measurer 800 and an upper arm cuff 500 which are connected to a surveillance monitor 700 and separated from each other, and the upper arm cuff 500 includes the air bag 310 and the pneumatic sensor 120.

The oxygen saturation measurer 800 measures the first arterial wave by using a sensor for measuring oxygen saturation, for example, an optical sensor (the first sensor 110), and the upper arm cuff 500 is a belt to be worn on the examinee's wrist and measures a variable pressure arterial wave (the second arterial wave) in the same manner as in the third embodiment described above by using the air bag and the pneumatic sensor provided in the upper arm cuff 500, that is, a cuff belt. That is, in the present embodiment, the upper arm cuff 500 includes an air bag and the second sensor but does not include the first sensor, and the oxygen saturation measurer functions as the first sensor.

In addition, when the first arterial wave and the second arterial wave (variable pressure arterial wave) are measured according to the fifth embodiment in the above-described manner, the blood pressure calculation unit 200 maps an arterial wave (the first arterial wave) measured under an isobaric pressure to an arterial wave (the second arterial wave) measured under a variable pressure based on an arterial wave block time when measuring the second arterial wave to calculate the mapped arterial wave, and calculates blood pressure by using the mapped arterial wave.

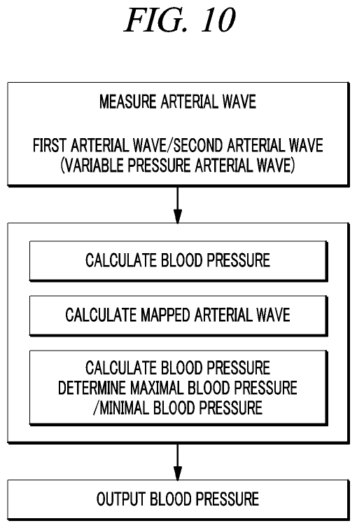

Referring to FIG. 10, an embodiment of a blood pressure measurement method performed by a blood pressure measurement system including a pulse wave measurement sensor unit for detecting an arterial wave includes a blood pressure calculation step of calculating a mapped arterial wave by mapping the first arterial wave measured under a constant pressure to the second arterial wave measured under a variable pressure by using a processor for calculating blood pressure, that is, the controller C, more specifically the blood pressure calculation unit 200, and calculating blood pressure by using the mapped arterial wave.

Calculation of the mapping arterial wave is performed based on an arterial wave block time when the second arterial wave is measured. In other words, in the present embodiment, a mapped arterial wave is calculated by mapping the first arterial wave measured under an isobaric pressure to the second arterial wave measured under the variable pressure, based on the arterial wave block time when the second arterial wave is measured, and blood pressure is calculated by using the mapped arterial wave.

In order to calculate the above-described blood pressure, an arterial wave measurement step of simultaneously measuring the first arterial wave and the second arterial wave at different regions of a human body by using the pulse wave measurement sensor unit 100 is performed.

In the arterial wave measurement step, the second arterial wave can be measured during a pressure increase process or a pressure reduction process of pressure of a region where the second arterial wave is measured. More specifically, in the arterial wave measurement step, the second arterial wave is measured during the pressure increase process or the pressure reduction process of the pressure of the region where the second arterial wave is measured.

In the blood pressure calculation step, the highest value of the mapped arterial wave is determined as a maximal blood pressure, and the lowest value of the mapped arterial wave is determined as a minimal blood pressure.

Referring to FIG. 11, a signal measured by the second sensor 120, for example, a variable pressure is converted into a pressure-to-variable pressure arterial wave (the second arterial wave), and the first sensor 110 measures an arterial wave at a constant pressure, that is, the first arterial wave.

The upper graph of the graphs illustrated in FIG. 11 illustrates pressure measured by the second sensor such as the pneumatic sensor described above during a pressure reduction process, for example, a process in which air filled in the air bag is exhausted, that is, the graph reflects pressure of the air bag itself and pressure of a blood vessel, and points a and b are points where the arterial wave is blocked.

In addition, a middle graph of the graphs illustrated in FIG. 11 illustrates a signal measured by the first sensor, that is, the first arterial wave.

Next, a lower graph of the graphs illustrated in FIG. 11 illustrates the mapped arterial wave described above, in which two graphs overlap each other such that the points a and b of the upper graph (the second arterial wave graph) overlap the same points in time (points c and d) of the middle graph (the first arterial wave graph). The highest value of the mapped arterial wave is determined as a maximal blood pressure, and the lowest value of the mapped arterial wave is determined as a minimal blood pressure. For reference, in mapping two arterial waves, an amplitude of the first arterial wave is corrected to accurately overlap the points c and d of the first arterial wave and the points a and b of the second arterial wave.

As described above, in the embodiment of the present invention, blood pressure is calculated by using a mapped arterial wave obtained by mapping a first arterial wave measured under an isobaric pressure to a variable pressure arterial wave measured under a variable pressure based on the first arterial wave and second arterial wave described above, and an arterial wave block point, more specifically, an arterial wave cutoff time is used as a mapping criterion.

More specifically, the controller C, particularly the blood pressure calculation unit 200 determines the highest value of the mapped arterial wave as a maximal blood pressure and the lowest value of the mapped arterial wave as a minimal blood pressure.

As such, the embodiments according to the present invention are described, and it is apparent to those skilled in the art that the present invention can be embodied in other specific forms without departing from the idea or scope in addition to the embodiments described above.

Therefore, the embodiments described above are to be regarded as illustrative rather than restrictive, and accordingly, the present invention is not limited to the above description and can be modified within the scope of the appended claims and their equivalents.

### INDUSTRIAL APPLICABILITY

The present invention is a blood pressure measurement device for measuring blood pressure of a human body, which can be used in the field of medical equipment, particularly in the field of blood pressure meter-related technology, and according to the present invention, a blood pressure value can be calculated quickly and accurately by using a signal detected for a short time.

1. A blood pressure measurement system comprising:
a pulse wave measurement sensor unit which measures arterial waves; and
a blood pressure calculation unit which calculates blood pressure from the arterial wave detected by the pulse wave measurement sensor unit,
wherein the pulse wave measurement sensor unit measures a first arterial wave under a constant pressure and measures a second arterial wave under a variable pressure, and
wherein the blood pressure calculation unit calculates a mapped arterial wave by mapping a first arterial wave measured under the constant pressure to a second arterial wave measured under the variable pressure and calculates blood pressure by using the mapped arterial wave.

2. The blood pressure measurement system of claim 1,
wherein the pulse wave measurement sensor unit includes a first sensor which measures the first arterial wave, and a second sensor which measures the second arterial wave.

3. The blood pressure measurement system of claim 2, further comprising:
a pressurization unit which applies pressure to a region where arterial wave measurement is performed by the second sensor.

4. The blood pressure measurement system of claim 3,
wherein the pressurization unit includes any one of a tightening device which tightens a portion to be tested, an air pump which injects air into an air bag, a thermal expansion member, and a shape memory alloy.

5. The blood pressure measurement system of claim 4,
wherein the pressurization unit further includes a valve which opens and closes at least one of a passage for guiding air to the air bag and an air outlet for discharging air from the air bag.

6. The blood pressure measurement system of claim 3,
wherein the second sensor measures the second arterial wave during one of a pressure increase process and a pressure reduction process.

7. The blood pressure measurement system of claim 2,
wherein each of the first sensor and the second sensor includes any one of a pressure sensor, an optical sensor, and an impedance sensor which measures impedance of a blood vessel.

8. The blood pressure measurement system of claim 7,
wherein the pressure sensor includes any one of a pneumatic sensor, a film-type pressure sensor, and a strain meter.

9. The blood pressure measurement system of claim 2,
wherein the first sensor and the second sensor respectively and simultaneously measures the first arterial wave and the second arterial wave at different positions.

10. The blood pressure measurement system of claim 1,
wherein the blood pressure calculation unit calculates the mapped arterial wave by mapping the first arterial wave to the second arterial wave based on an arterial wave block time when the second arterial wave is measured.

11. The blood pressure measurement system of claim 10,
wherein the blood pressure calculation unit determines a highest value of the mapped arterial wave as a maximal blood pressure and determines a lowest value of the mapped arterial wave as a minimal blood pressure.

12. A blood pressure measurement method performed by a blood pressure measurement system including a pulse wave measurement sensor unit for detecting an arterial wave, the blood pressure measurement method comprising:
a blood pressure calculation step of calculating a mapped arterial wave by mapping a first arterial wave measured under an isobaric pressure to a second arterial wave measured under a variable pressure by using a processor for calculating blood pressure, and calculating the blood pressure from the mapped arterial wave by using the processor.

13. The blood pressure measurement method of claim 12, further comprising:
an arterial wave measurement step of simultaneously measuring the first arterial wave and the second arterial wave at different positions by using the pulse wave measurement sensor unit.

14. The blood pressure measurement method of claim 13,
wherein, in the arterial wave measurement step, the second arterial wave is measured during one of a pressure increase process and a pressure reduction process of a region where the second arterial wave is measured.

15. The blood pressure measurement method of claim 12,
wherein, in the blood pressure calculation step, the mapped arterial wave is calculated by mapping the first arterial wave to the second arterial wave based on an arterial wave block time when the second arterial wave is measured.

16. The blood pressure measurement method of claim 15,
wherein, in the blood pressure calculation step, a highest value of the mapped arterial wave is determined as a maximal blood pressure, and a lowest value of the mapped arterial wave is determined as a minimal blood pressure.
